(19) United States
(12) Patent Application Publication
(10) Pub. No.: US 2016/0089062 A1
(43) Pub. Date: Mar. 31, 2016

(54) METHOD AND A MOBILE APP FOR USE WITH A MOBILE DEVICE TO ACCURATELY QUANTIFY BLOOD HEMOGLOBIN IN MAMMALS

(71) Applicants: Yudaya R. Sivathanu, West Lafayette, IN (US); Jongmook Lim, West Lafayette, IN (US)

(72) Inventors: Yudaya R. Sivathanu, West Lafayette, IN (US); Jongmook Lim, West Lafayette, IN (US)

(21) Appl. No.: 14/503,379

(22) Filed: Sep. 30, 2014

(51) Int. Cl. A61B 5/1455; A61B 5/145; A61B 19/00; A61B 3/00; A61B 3/12; A61B 5/1495; A61B 3/14; A61B 5/00

(52) U.S. Cl. CPC A61B 5/1455; A61B 3/145; A61B 5/14546; A61B 5/7278; A61B 3/0025; A61B 5/748; A61B 3/1241; A61B 5/6898; A61B 5/1495; A61B 19/5212; A61B 2576/02; A61B 2503/40; A61B 2562/0233

(57) ABSTRACT

A method and a mobile app are disclosed for obtaining hemoglobin count in the blood of a mammal using digital images of the conjunctiva of the eye of the mammal taken with a camera of a mobile device running the mobile app. The mobile app carries out a method including: obtaining a color image of the conjunctiva of the eye using the digital camera; computing an R/B ratio and a Y/I ratio of the color image; normalizing the R/B ratio and the Y/I ratio using color image normalization parameters obtained using a master camera so as to provide a R/B true ratio and a Y/I true ratio; and using the R/B true ratio and the Y/I true ratio as inputs to respective look-up tables created using the master camera so as to provide an accurate measure of blood hemoglobin count.

### FIELD OF THE INVENTION:

This invention relates generally to systems for measurement of blood hemoglobin concentration, and particularly to devices and methods for optical measurement of blood hemoglobin concentration.

### BACKGROUND OF THE INVENTION:

Anemia results in pale mucous membranes and conjunctiva in many mammalian species. Currently, there are several non-invasive optical devices on the market that measure blood hemoglobin concentration (also called hemoglobin count). At present, these devices have not been widely adopted. The primary reasons for the lack of wide-spread use of these devices include their inconvenient size and poor portability (devices based on the NIR (near infra-red) spectrum), difficulty interpreting the results, and poor correlation between noninvasive and laboratory measures of blood hemoglobin concentration (e.g., multi-wavelength pulse oximeters). A visual color scale, such as the FAMACHA system, is widely used in small ruminant production. However, use of this visual color scale involves a subjective measure that is subject to operator error, and can only deliver a qualitative and/or unreliable estimate of anemia.

### SUMMARY OF THE INVENTION

The invention provides a convenient, objective, and quantitative measure of blood hemoglobin using a non-invasive optical method, including acquiring an image using a built-in camera of a smart phone or tablet computing device, for example, and processing the image so as to provide a value that accurately represents blood hemoglobin concentration. These functions can advantageously be incorporated into an app running on the smartphone or tablet to conveniently and accurately determine blood hemoglobin concentration in any mammal, such as a human.

A general aspect of the invention is a method for obtaining blood hemoglobin count using images of the conjunctiva of an eye of a mammal, the images being obtained using a digital camera. The method includes: obtaining a color image of the conjunctiva of an eye of a mammal using a digital camera; computing an R/B ratio and a Y/I ratio of the color image; normalizing the R/B ratio and the Y/I ratio using color image normalization parameters obtained using a master camera so as to provide a R/B true ratio and a Y/I true ratio; and using the R/B true ratio and the Y/I true ratio as inputs to respective look-up tables created using the master camera so as to provide a measure of blood hemoglobin count.

In some embodiments, computing an R/B ratio and a Y/ ratio of the color image includes: choosing a plurality of analysis areas of the color image that are fully within the conjunctiva of the mammal's eye; computing an R/B ratio and a Y/I ratio for each of the analysis areas; and computing an average R/B ratio and an average Y/I ratio so as to provide the R/B ratio and the Y/I ratio used for normalizing.

In some embodiments, the the look-up table includes a set of empirically determined data points, and a set of interpolated data points derive from the set of empirically determined data points.

In some embodiments, the look-up table is converted to a curve that is fit to the set of empirically determined data points, and the R/B true ratio and the Y/I true ratio are used to find a corresponding blood hemoglobin count.

In some embodiments, using the R/B true ratio and the Y/I true ratio as inputs to respective look-up tables created using the master camera provides two respective values of blood hemoglobin count that are averaged so as to provide an accurate measure of blood hemoglobin count.

In some embodiments, normalizing the R/B ratio and the Y/I ratio using color image normalization parameters obtained using a master camera so as to provide a R/B true ratio and a Y/I true ratio is performed as follows: R/B true ratio=(R/B ratio)/(R/B master camera ratio); and Y/I true ratio=(Y/I ratio)/(Y/I master camera ratio).

In some embodiments, the digital camera is a camera of one of: a mobile phone, a tablet computing device, a portable digital camera.

Another general aspect of the invention is a mobile app for storage and use on a mobile device, the mobile device having a digital camera and a non-transitory computer-readable medium for storing the mobile app, the mobil app being for obtaining hemoglobin count in blood using images of the conjunctiva of the eye of a mammal. The mobile app carries out a method including: obtaining a color image of the conjunctiva of an eye of a mammal using a digital camera of the mobile device; computing an R/B ratio and a Y/I ratio of the color image; normalizing the R/B ratio and the Y/I ratio using color image normalization parameters obtained using a master camera so as to provide a R/B true ratio and a Y/I true ratio; and using the R/B true ratio and the Y/I true ratio as inputs to respective look-up tables created using the master camera so as to provide a measure of blood hemoglobin count.

In some embodiments, computing an R/B ratio and a VI ratio of the color image includes: choosing a plurality of analysis areas of the color image that are fully within the conjunctiva of the mammal's eye; computing an R/B ratio and a Y/I ratio for each of the analysis areas; and computing an average R/B ratio and an average Y/I ratio so as to provide the R/B ratio and the Y/I ratio used for normalizing.

In some embodiments, the the look-up table includes a set of empirically determined data points, and a set of interpolated data points derive from the set of empirically determined data points.

In some embodiments, the look-up table is converted to a curve that is fit to the set of empirically determined data points, and the R/B true ratio and the Y/I true ratio are used to find a corresponding blood hemoglobin count.

In some embodiments, using the R/B true ratio and the Y/I true ratio as inputs to respective look-up tables created using the master camera provides two respective values of blood hemoglobin count that are averaged so as to provide an accurate measure of blood hemoglobin count.

In some embodiments, normalizing the R/B ratio and the Y/I ratio using color image normalization parameters obtained using a master camera so as to provide a R/B true ratio and a Y/I true ratio is performed as follows: R/B true ratio=(R/B ratio)/(R/B master camera ratio); and Y/I true ratio=(Y/I ratio) (Y/I master camera ratio).

Another general aspect of the invention is a computer program product for obtaining hemoglobin count in blood using images of the conjunctiva of the eye of a mammal, wherein the computer program product includes computer code that can be stored on a non-transitory computer-readable medium of a computing device, the computer code capable of being accessed and executed by the computing device to perform a method including: obtaining a color image of the conjunctiva of an eye of a mammal using a digital camera; computing an R/B ratio and a Y/I ratio of the color image; normalizing the R/B ratio and the Y/I ratio using color image normalization parameters obtained using a master camera so as to provide a R/B true ratio and a Y/I true ratio; and using the R/B true ratio and the Y/I true ratio as inputs to respective look-up tables created using the master camera so as to provide a measure of blood hemoglobin count.

In some embodiments, computing an R/B ratio and a Y/I ratio of the color image includes: choosing a plurality of analysis areas of the color image that are fully within the conjunctiva of the mammal's eye; computing an R/B ratio and a Y/I ratio for each of the analysis areas; and computing an average R/B ratio and an average Y/I ratio so as to provide the R/B ratio and the Y/I ratio used for normalizing.

In some embodiments, the the look-up table includes a set of empirically determined data points, and a set of interpolated data points derive from the set of empirically determined data points.

In some embodiments, the look-up table is converted to a curve that is fit to the set of empirically determined data points, and the R/B true ratio and the Y/I true ratio are used to find a corresponding blood hemoglobin count.

In some embodiments, using the R/B true ratio and the Y/I true ratio as inputs to respective look-up tables created using the master camera provides two respective values of blood hemoglobin count that are averaged so as to provide an accurate measure of blood hemoglobin count.

In some embodiments, normalizing the R/B ratio and the Y/I ratio using color image normalization parameters obtained using a master camera so as to provide a R/B true ratio and a Y/I true ratio is performed as follows: R/B true ratio=(R/B ratio)/(R/B master camera ratio); and Y/I true ratio=(Y/I ratio)/(Y/I master camera ratio).

In some embodiments, the digital camera is a camera of one of: a mobile phone, a tablet computing device, a portable digital camera.

### BRIEF DESCRIPTION OF THE DRAWINGS

The invention will be more fully understood by reference to the detailed description, in conjunction with the following figures, wherein:

FIG. 1 is a schematic diagram of a calibration system of the invention showing use of the Master Camera.

FIG. 2 is graph showing correlation of R/G and R/B intensities with hemoglobin count.

FIG. 3 is a graph showing correlation of Y/ and Q/I with hemoglobin count.

FIG. 4A is an image of a cow's eye showing a small region to be used for analysis.

FIG. 4B is a close-up of the small region shown in FIG. 4A.

### DETAILED DESCRIPTION

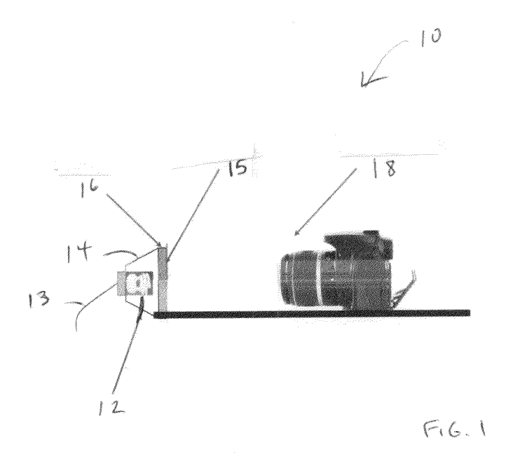

With reference to FIG. 1, an apparatus for calibration of cameras, including mobile phone cameras and other cameras, calibration tool 10, is shown in FIG. 1 (manufactured by En'Urga Inc).

The LED lamp 12 of the calibration tool 10 is a full spectrum lamp, placed inside a reflector 14 with a light diffuser 16 (e.g. Fusion Optix(™)) so as to provide a relatively uniform back ground. The lamp 12 is energized by the power cord 13. The camera 18 is focused onto the front-facing surface of the diffuser 16, which serves as a flat screen. Full-spectrum images of the flat screen are obtained. Next, filtered images of the flat screen are obtained using three plastic sheet filters (red, blue, and green) placed sequentially over the flat screen.

Once these filtered images have been obtained, color image normalization parameters are calculated in several different color spaces such as RGB, YIQ, YUV, etc. Both the YUV and YIQ color spaces are intended to take advantage of human color response characteristics.

For instance, the YUV color space is defined in terms of one luminance (Y) component and two chrominance (UV) components. The primary advantage of defining the image in the YUV space is that it reduces the bandwidth of the image, and more closely corresponds to the human eye perception that has a lower color depth. In addition, the YUV color space distinguishes luminance with greater resolution, and represents chrominance with an orthogonal relationship.

The transformation matrix from RGB to YUV color space is:

$$[ \begin{matrix}Y \\ U \\ V\end{matrix} ] = [ \begin{matrix}0.299 & 0.587 & 0.114 \\ - .147 & - .289 & 0.436 \\ 0.615 & - .515 & - .100\end{matrix} ] [ \begin{matrix}R \\ G \\ B\end{matrix} ] \qquad ( 1 )$$

After U and V are obtained, the chromo cue (a 2-D vector denoting displacement C, and amplitude θ, can be obtained as:

c=\sqrt{|u|²+|v|²} and θ=tan⁻¹(v/u) (2)

The YIQ color space is very similar to the YUV color space. Y as before is the luminance, where I stands for in-phase, and Q stands for quadrature, referring to the components used in a quadrature amplitude modulation scheme. The YIQ system is intended to take advantage of human color response characteristics. The eye is more sensitive to changes in the orange-blue (I) range than in the purple-green range (Q)—therefore less bandwidth is required for Q than for I.

In the YUV color space, the U and V components can be thought of as X and Y coordinates within the color space. I and Q can be thought of as a second pair of axes on the same graph, rotated 33°; therefore IQ and UV represent different coordinate systems on the same plane.

In the YUV system, since U and V both contain information in the orange-blue range, both components must be given the same amount of bandwidth as the amount of bandwidth given to I so as to achieve similar color fidelity.

The transformation matrix from RGB to YIQ color space is as follows:

$$[ \begin{matrix}Y \\ I \\ Q\end{matrix} ] = [ \begin{matrix}0.299 & 0.587 & 0.114 \\ 0.596 & - .274 & - .322 \\ - .212 & - .523 & 0.311\end{matrix} ] [ \begin{matrix}R \\ G \\ B\end{matrix} ] \qquad ( 3 )$$

So, in addition to the values of RGB, several additional variables such as Y, U, V, I, are obtained for the individual camera.

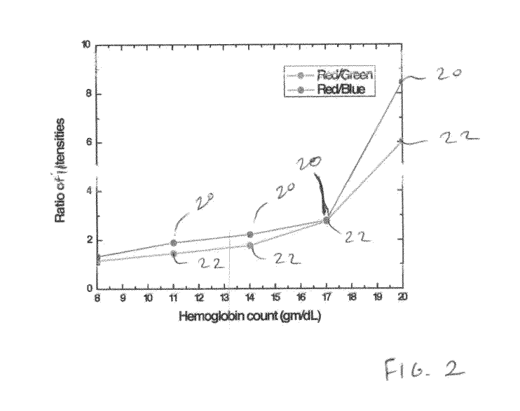

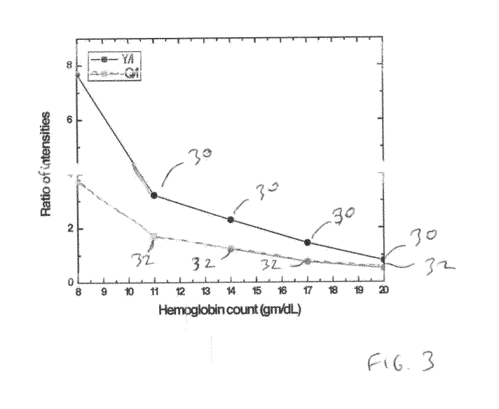

The hemoglobin concentration is directly correlated to ratios (or averages of ratios) of several of these variables. Two correlations are shown in FIG. 2 and FIG. 3, respectively.

With reference to FIG. 2, data points 20 that correlate specific values of Red/Blue ratios of intensities of with Hemoglobin count in gm/dL are shown plotted and connected to neighboring values 20. Likewise, data points 22 that correlate specific values of Red/Green ratios of intensities of with Hemoglobin count in gm/dL are shown plotted and connected to neighboring values 22.

With reference to FIG. 3, analogously, data points 30 that correlate specific values of Y/I ratios of intensities of with Hemoglobin count in gm/dL are shown plotted and connected to neighboring values 30. Likewise, data points 32 that correlate specific values of Q/I ratios of intensities of with Hemoglobin count in gm/dL are shown plotted and connected to neighboring values 32.

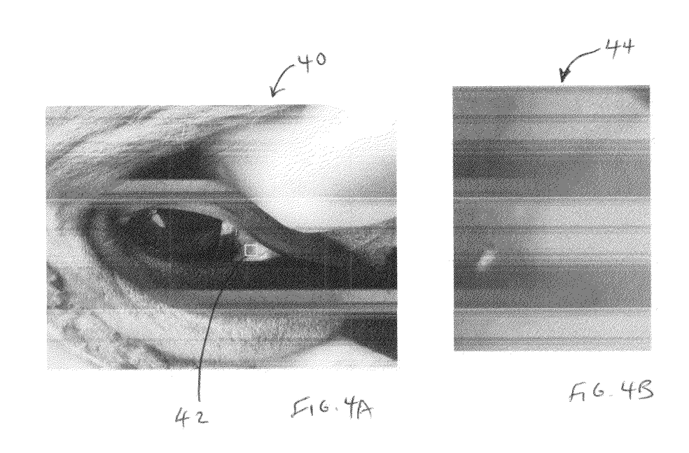

Referring to FIG. 4A, once the camera 18 is calibrated using the calibration tool 10 any camera (in a mobile phone or in a stand-alone digital camera) can be used to take an image 40 of the eye of a cow, or any other mammal (e.g., a person). As soon as the image 40 of the eye is obtained, the user can enlarge the image 40 and then select a portion 42 of the image of mucosa (shown in the yellow square in FIG. 4) for analysis. A magnified view 44 of the selected portion 42 is shown in FIG. 4B.

The selected portion 42 of the image 40 is then used to obtain ratios of R/B, R/G, Y/I, Y/Q. Then, the ratios R/B, R/G, Y/I, Y/Q are used as input to the correlation curves (or lookup tables) to determine the hemoglobin count in the blood.

Detailed Steps for Determining a Calibration Function for Determining Hemoglobin Blood Count (HBC) in Blood

Step 1: Referring to FIG. 1, choose a master camera 18, and place it at a specific distance (approximately 10 inches) from a pure spectrum light source 12 (such as an MR11 LED lamp). If the light source 12 does not have a diffuser 16 in front of it, then place a piece of white paper in between the lamp 12 and the camera 18 to act as a diffuser. First place a red filter 15 in front of the diffuser 16. Try to get a uniform color in the picture. Take a red-filtered image. Then replace the red filter with a blue filter. Take a blue-filtered image. Then replace the blue filter with a green filter. Take a green-filtered image. From the red-filtered, blue-filtered, and green-filtered images, compute the mean RGB values of the master camera 18, i.e., Rₘₑₐₙ, Gₘₑₐₙ, Bₘₑₐₙ. Compute the ratio of Rₘₑₐₙ over Bₘₑₐₙ, and call it “R/B master”.

Transform the images from the RGB space to the YIQ space, as described herein above using Equation 3. Compute the ratio Y/I as above, and call it “Y/I master”, These are the master ratios R/B and Y/I of the specific filters that are used as color image normalization parameters R/B master and Y/I master for normalizing the images taken by any digital camera, such as the built-in digital camera of a smart phone or other mobile device.

Step 2: Take an image 40 of the mammal's eye with the master camera 18. Use the manual zoom feature of the camera 18 and select two or three relatively uniform areas 42 of the conjunctiva of the mammal's eye. Mark those areas of the picture 40 as the analysis areas 42.

Step 3: Compute the mean (or the Median, or any quantile thereof) R, G, and B values for each area 42 so as to compute the R/G ratio and the R/B ratio for the selected area 42. Transform the RGB image into Y/Q space using Equation 3. Obtain the Y/I and Q/I ratio values for the same areas 42.

Step 4: Using conventional laboratory methods, obtain the hemoglobin blood count of the mammal for different hemoglobin blood counts all the way from acute anemia to normal.

Step 5: Using the master camera 18, obtain several images of the mammal conjunctiva for different hemoglobin blood counts all the way from acute anemia to normal with he master camera 18. Obtain the analogous R/G and R/B ratios for all the images. Obtain the corresponding Y/I and Q/I ratios for these images.

Step 6: Plot each measured R/B ratio (e.g., as in FIG. 2) and the corresponding Y/I ratios (e.g. as in FIG. 3) of the images as a function of the hemoglobin blood count.

Step 7: Obtain an interpolated correlation among the measured R/B ratios and hemoglobin count, and an interpolated correlation among the corresponding Y/I ratios and hemoglobin blood count using curve fitting, so ANY R/B ratio and ANY Y/I ratio can be used to determine a corresponding hemoglobin count (as shown in FIGS. 2 and 3).

Detailed Steps for Using the Calibration Function with other Cameras, Such as a Smartphone Camera, for Determining Hemoglobin Blood Count (HBC) in Blood

Step 8: For a digital camera that is NOT the master camera (such as the built-in digital camera of a smart phone or of a tablet computer), place the digital camera in front of the same filters 15 as were used with the master camera 18.

Step 9: Obtain the R/B ratio and the Y/I ratio for each of the R, G, and B filters for he digital camera, as in Step 1.

Step 10: Normalize the R/B ratio and Y/I ratio of the digital camera to the R/B ratio and Y/I ratio of the master camera to create the R/B true ratio and the Y/I true ratio as follows:

R/B true ratio=(R/B digital camera ratio)/(R/B master camera ratio)

Y/I true ratio=(Y/I digital camera ratio)/(Y/I master camera ratio)

Step 11: Acquire an image of the mammal's eye using the digital camera and choose two or three analysis areas that are fully within the conjunctiva of the mammal's eye.

Step 12; Obtain the average R/B digital camera ratio averaged over the two or three analysis areas, and the average Y/I digital camera ratio averaged over the two or three analysis areas, as in Step 3. Using these averages, normalize them using the formulae in Step 10 to get the R/B true ratio and the Y/I true ratio.

Step 13: Go to the interpolated curves (as in Step 7) (or an analogous look-up table), and use the R/B true ratio on the vertical axis to obtain a corresponding R/B hemoglobin blood count on the horizontal axis using the R/B interpolated curve (FIG. 2), and also use the Y/I true ratio on the vertical axis to obtain a corresponding Y/I hemoglobin blood count on the horizontal axis using the Y/I interpolated curve (FIG. 3).

Step 14: Compute the average of the R/B hemoglobin blood count and the Y/I hemoglobin blood count to obtain an accurate hemoglobin blood count.

Other modifications and implementations will occur to those skilled in the art without departing from the spirit and the scope of the invention as claimed. Accordingly, the above description is not intended to limit the invention except as indicated in the following claims.

What is claimed is:

1. A method for obtaining blood hemoglobin count using images of the conjunctiva of an eye of a mammal, the images being obtained using a digital camera, the method comprising:
obtaining a color image of the conjunctiva of an eye of a mammal using a digital camera;
computing an R/B ratio and a Y/I ratio of the color image;
normalizing the R/B ratio and the Y/I ratio using color image normalization parameters obtained using a master camera so as to provide a R/B true ratio and a Y/I true ratio; and
using the R/B true ratio and the Y/I true ratio as inputs to respective look-up tables created using the master camera so as to provide a measure of blood hemoglobin count.

2. The method of claim 1, wherein computing an R/B ratio and a Y/I ratio of the color image includes:
choosing a plurality of analysis areas of the color image that are fully within the conjunctiva of the mammal's eye;
computing an R/B ratio and a Y/I ratio for each of the analysis areas; and
computing an average R/B ratio and an average Y/I ratio so as to provide the R/B ratio and the Y/I ratio used for normalizing.

3. The method of claim 1, wherein the the look-up table includes a set of empirically determined data points, and a set of interpolated data points derive from the set of empirically determined data points.

4. The method of claim 1, wherein the look-up table is converted to a curve that is fit to the set of empirically determined data points, and the R/B true ratio and the Y/I true ratio are used to find a corresponding blood hemoglobin count.

5. The method of claim 1, wherein using the R/B true ratio and the Y/I true ratio as inputs to respective look-up tables created using the master camera provides two respective values of blood hemoglobin count that are averaged so as to provide an accurate measure of blood hemoglobin count.

6. The method of claim 1, wherein normalizing the R/B ratio and the WI ratio using color image normalization parameters obtained using a master camera so as to provide a R/B true ratio and a Y/I true ratio is performed as follows:
R/B true ratio=(R/B ratio)/(R/B master camera ratio); and
Y/I true ratio=(Y/I ratio)/(Y/I master camera ratio).

7. The method of claim 1, wherein the digital camera is a camera of one of:
a mobile phone, a tablet computing device, a portable digital camera.

8. A mobile app for storage and use on a mobile device, the mobile device having a digital camera and a non-transitory computer-readable medium for storing the mobile app, the mobil app being for obtaining hemoglobin count in blood using images of the conjunctiva of the eye of a mammal, the mobile app carrying out a method comprising:
obtaining a color image of the conjunctiva of an eye of a mammal using a digital camera of the mobile device;
computing an R/B ratio and a Y/I ratio of the color image;
normalizing the R/B ratio and the Y/I ratio using color image normalization parameters obtained using a master camera so as to provide a R/B true ratio and a WI true ratio; and
using the R/B true ratio and the Y/I true ratio as inputs to respective look-up tables created using the master camera so as to provide a measure of blood hemoglobin count.

9. The mobile app of claim 8, wherein computing an R/B ratio and a Y/I ratio of he color image includes:
choosing a plurality of analysis areas of the color image that are fully within the conjunctiva of the mammal's eye;
computing an R/B ratio and a Y/I ratio for each of the analysis areas; and
computing an average R/B ratio and an average Y/I ratio so as to provide the R/B ratio and the Y/I ratio used for normalizing.

10. The mobile app of claim 8, wherein the the look-up table includes a set of empirically determined data points, and a set of interpolated data points derive from the set of empirically determined data points.

11. The mobile app of claim 8, wherein the look-up table is converted to a curve that is fit to the set of empirically determined data points, and the R/B true ratio and the Y/I true ratio are used to find a corresponding blood hemoglobin count.

12. The mobile app of claim 8, wherein using the R/B true ratio and the Y/I true ratio as inputs to respective look-up tables created using the master camera provides two respective values of blood hemoglobin count that are averaged so as to provide an accurate measure of blood hemoglobin count.

13. The mobile app of claim 8, wherein normalizing the R/B ratio and the Y/I ratio using color image normalization parameters obtained using a master camera so as to provide a R/B true ratio and a Y/I true ratio is performed as follows:
R/B true ratio=(R/B ratio)/(R/B master camera ratio); and
Y/I true ratio=(Y/I ratio)/(Y/I master camera ratio).

14. A computer program product for obtaining hemoglobin count in blood using images of the conjunctiva of the eye of a mammal, wherein the computer program product includes computer code that can be stored on a non-transitory computer-readable medium of a computing device, the computer code capable of being accessed and executed by the computing device to perform a method comprising:
obtaining a color image of the conjunctiva of an eye of a mammal using a digital camera;
computing an R/B ratio and a Y/I ratio of the color image;
normalizing the R/B ratio and the Y/I ratio using color image normalization parameters obtained using a master camera so as to provide a R/B true ratio and a Y/I true ratio; and
using the R/B true ratio and the Y/I true ratio as inputs to respective look-up tables created using the master camera so as to provide a measure of blood hemoglobin count.

15. The computer program product of claim 14, wherein computing an R/B ratio and a Y/I ratio of the color image includes:
choosing a plurality of analysis areas of the color image that are fully within the conjunctiva of the mammal's eye;
computing an R/B ratio and a Y/I ratio for each of the analysis areas; and
computing an average R/B ratio and an average Y/I ratio so as to provide the R/B ratio and the Y/I ratio used for normalizing.

16. The computer program product of claim 14, wherein the the look-up table includes a set of empirically determined data points, and a set of interpolated data points derive from the set of empirically determined data points.

17. The computer program product of claim 14, wherein the look-up table is converted to a curve that is fit to the set of empirically determined data points, and the R/B true ratio and the Y/I true ratio are used to find a corresponding blood hemoglobin count.

18. The computer program product of claim 14, wherein using the R/B true ratio and the Y/I true ratio as inputs to respective look-up tables created using the master camera provides two respective values of blood hemoglobin count that are averaged so as to provide an accurate measure of blood hemoglobin count.

19. The computer program product of claim 14, wherein normalizing the R/B ratio and the Y/I ratio using color image normalization parameters obtained using a master camera so as to provide a R/B true ratio and a Y/I true ratio is performed as follows:
R/B true ratio=(R/B ratio)/(R/B master camera ratio); and
Y/I true ratio=(Y/I ratio)/(Y/I master camera ratio).

20. The computer program product of claim 14, wherein the digital camera is a camera of one of: a mobile phone, a tablet computing device, a portable digital camera.
